(19) United States
(12) Patent Application Publication
(10) Pub. No.: US 2026/0199387 A1
(43) Pub. Date: Jul. 16, 2026

(54) ARMED CAR-MACROPHAGE COMPOSITIONS AND METHODS FOR THERAPY OF HEPATOCELLULAR CARCINOMA

(71) Applicant: RenaBio Therapeutics, Inc., Natick, MA (US)

(72) Inventors: Shengjun Ren, Newton, MA (US); Yu Zhang, Newton, MA (US)

(21) Appl. No.: 19/448,660

(22) Filed: Jan. 14, 2026

Related U.S. Application Data
(60) Provisional application No. 63/745,158, filed on Jan. 14, 2025.

(51) Int. Cl. A61K 31/7088; A61K 9/00; A61K 9/51; A61K 47/24; A61K 47/28; A61P 35/00; C07K 16/22; C12N 15/11

(52) U.S. Cl. CPC A61K 31/7088; A61K 9/0019; A61K 9/5123; A61K 47/24; A61K 47/28; A61P 35/00; C07K 16/22; C12N 15/11

(57) ABSTRACT

A nanoparticle for treating hepatocellular carcinoma (HCC) in which the nanoparticle includes a lipid phase and a core that contains one or more nucleic acids encoding a chimeric antigen receptor (CAR), a T-cell engager (TCE), and/or a therapeutic gene. The CAR and the TCE each binding specifically to an HCC tumor-associated antigen and the nanoparticle selectively delivers the one or more nucleic acids to M2 macrophages in vitro and in vivo. Also provided is a therapeutic composition that contains a plurality of the nanoparticle described above and a pharmaceutically acceptable excipient. Further provided are methods for converting HCC tumor-resident M2 macrophages into M1 macrophages, and for treating HCC.

### CROSS REFERENCE TO RELATED APPLICATIONS

This application claims the priority of U.S. Provisional Application Ser. No. 63/745,158, filed Jan. 14, 2025, the content of which is hereby incorporated by reference in its entirety.

### REFERENCE TO AN ELECTRONIC SEQUENCE LISTING

A computer readable file containing a sequence listing is being electronically co-filed herewith via Patent Center. The computer readable file, submitted under 37 CFR § 1.831 (e), will also serve as the copy required by 37 § CFR 1.831 (c). The file (filename “RENA-002-101X.XML”) was created on Jan. 3, 2026, and has a size of 19,193 bytes. The content of the computer readable file is hereby incorporated by reference in its entirety.

### BACKGROUND

Adoptive cell therapy, such as chimeric antigen receptor (CAR) T cell therapy, has demonstrated great therapeutic efficacy in hematological malignancies. However, it has major drawbacks in treating solid tumors, in part, due to tumor microenvironment (TME) immunosuppression and antigen escape.

CAR T cell therapy requires a complicated and lengthy ex vivo manufacturing process. The extremely high cost of the therapy limits its accessibility to patients.

There is a critical need to develop adoptive cell therapy alternatives to traditional CAR T cell therapy, particularly for treating solid tumors such as hepatocellular carcinoma, in the form of an off-the-shelf therapeutic product that has a lower cost and increased functionality that overcomes the drawbacks mentioned above.

### SUMMARY

To meet the above challenges, armed CAR-macrophage cell therapy is disclosed whose CAR gene, together with armed therapeutic modality genes are transferred into macrophages using an in vitro or in vivo (in situ) targetable nanoparticle to treat hepatocellular carcinoma (HCC).

To accomplish this therapy, a nanoparticle for treating HCC is disclosed. The nanoparticle includes a lipid phase and a core that contains one or more nucleic acids. The core contains one or more nucleic acids that encode a chimeric antigen receptor (CAR), a T-cell engager (TCE), and/or a therapeutic gene. The CAR and the TCE each bind specifically to an HCC tumor-associated antigen The nanoparticle selectively delivers the one or more nucleic acids to M2 macrophages in vitro and in vivo.

Also provided is a therapeutic composition that contains a plurality of the nanoparticle described above and a pharmaceutically acceptable excipient.

Further provided is a method for converting HCC tumor-resident M2 macrophages into M1 macrophages by contacting the M2 macrophages with a plurality of the nanoparticle set forth, supra.

Moreover, disclosed is a method for treating HCC in a subject. The method comprising administering to the subject a composition comprising the nanoparticle mentioned above in which the one or more nucleic acids encode (i) a CAR that specifically binds to GPC-3, (ii) a TCE that specifically binds to another HCC TAA and binds to a T cell, and (iii) a therapeutic gene that is effective against the HCC.

The details of one or more embodiments of the invention are set forth in the description and drawings below. Other features, objects, and advantages of the invention will be apparent from the description, the drawings, and the claims.

### BRIEF DESCRIPTION OF THE DRAWINGS

The foregoing and other objects, features and advantages will be apparent from the following description of particular embodiments of the disclosure, as illustrated in the accompanying drawings.

FIG. 1 shows bar graphs of luciferase activity (expressed as tumor burden relative to M2 macrophage control) of hepatocellular carcinoma (HCC) HepG2 cells stably expressing luciferase and green fluorescent protein (HepG2_GFP/Luci) co-cultured with M2 macrophages untreated (M2) or treated with 1 μg/ml of the indicated CAR mRNA LNP. The luciferase activities were measured 1, 2, or 3 days after initiation of treatment. E/T=effector (M2) to target (HCC) ratio.

FIG. 2 shows bar graphs of Tumor burden relative to HepG2 control of HepG2_GFP/Luci cells co-cultured with M2 macrophages untreated (M2) or treated for 1 day with the indicated concentration of CAR mRNA LNP constructs at an E/T of 1/8.

FIG. 3 shows fluorescence microscopy images (upper panels) and corresponding phase contrast images (lower panels) of HepG2_GFP/Luci cells co-cultured with M2 macrophages untreated (left panels) or treated for 1 day with 1.5 μg/ml CAR^{SR9} mRNA LNP (right panels) at an E/T of 1/8.

FIG. 4 shows fluorescence microscopy images (upper panels) and corresponding phase contrast images (lower panels) of HepG2_GFP/Luci cells co-cultured with M2 macrophages untreated (left panels) or treated for 1 day with 3.0 μg/ml CAR^{SR9} mRNA LNP (right panels) at an E/T of 1/8.

FIG. 5 shows fluorescence microscopy images (upper panels) and corresponding phase contrast images (lower panels) of HepG2_GFP/Luci cells co-cultured with M2 macrophages untreated (left panels) or treated for 1 day with 6.0 g/ml CAR^{SR9} mRNA LNP (right panels) at an E/T of 1/8.

FIG. 6 shows bar graphs of relative light units (RLU) of NFAT-Jurkat T cells (NFAT; T cells expressing luciferase under the control of the NFAT promoter) co-cultured with HepG2 cells and treated with supernatant from M2 macrophages (M2 control) or supernatant from M2 macrophages transfected with the indicated T cell engager mRNA LNP construct (TCE). Supernatant from transfected M2 was collected 1, 2, 5, or 7 days after TCE mRNA LNP treatment. Luciferase activities were measured 12 h after treatment. Background luciferase activity was measured from cultures of NFAT, HepG2, co-cultured NFAT and HepG2 (NFAT+HepG2), and medium alone.

FIG. 7 shows bar graphs as described in the legend for FIG. 6 except that the luciferase activity of NFAT-Jurkat cells co-cultured with HepG2 cells was measured 24 h after treatment.

FIG. 8 shows bar graphs of killing percentage of HepG2_GFP/Luci cells cocultured with T cells and treated with supernatant collected from M2 macrophages 1, 2, 5, or 7 days after transfection with the indicated TCE mRNA LNP. Killing percentage was calculated from luciferase activities of the indicated cultures compared to luciferase activity of HepG2_GFP/Luci cells cocultured with T cells treated with supernatant from untransfected M2 macrophages (0% killing). Luciferase activities were measured 48 h after treatment. The E/T ratio of the effective primary T cells to the targeting HCC HepG2 cancer cells was 1/2.

FIG. 9 shows bar graphs as described in the legend for FIG. 8 except that the luciferase activities were measured 72 h after treatment.

FIG. 10 shows bar graphs of luciferase activity (expressed as tumor burden compared to HepG2_GFP/Luci plus M2) of HepG2_GFP/Luci cells co-cultured with M2 macrophages and treated for 48 h with T cells and 1.0 μg/ml CAR^{SR9} mRNA LNP (CAR LNP) or 0.5 μg/ml TCE^{SR4} and CAR^{SR9} mRNA LNP (TCE_CAR LNP). The E/T for M2 was 1/8 and for T cells 1/4.

FIG. 11 shows bar graphs of luciferase activity (expressed as tumor burden relative to HepG2_GFP/Luci) of HepG2_GFP/Luci cells alone (HepG2) or HepG2_GFP/Luci cells co-cultured with M2 and treated for 24 h with T cells (Pan-T) alone (control) or T cells with the indicated amounts of TCE^{SR4} mRNA LNP (TCE_SR4) or TCE^{SR4}-CAR^{SR9} mRNA LNP (TCE_SR4-CAR_SR9). The E/T for M2 was 1/8 and for T cells 1/4.

FIG. 12 shows bar graphs of luciferase activity (expressed as tumor burden relative to HepG2_GFP/Luci+M2+T) of HepG2_GFP/Luci cells co-cultured with M2 and treated with T cells alone (control) or T cells with 2 μg/ml TCE^{SR4} mRNA LNP (TCE LNP) or 2 μg/ml TCE^{SR4}-CAR^{SR9} mRNA LNP (TCE-CAR LNP). Luciferase activity was measured 7 days after treatment. The E/T for M2 was 1/64 and for T cells 1/32.

FIG. 13 shows fluorescence microscopy images (upper panels) and corresponding phase contrast images (lower panels) of HepG2_GFP/Luci cells co-cultured with M2 macrophages and treated with T cells (left panels) or treated with T cells and 1.0 g/ml TCE^{SR4}-CAR^{SR9} mRNA LNP (right panels). Images were obtained 3 days after treatment. The E/T for M2 was 1/8 and for T cells 1/2.

FIG. 14 shows fluorescence microscopy images (upper panels) and corresponding phase-contrast images (lower panels) obtained before and 6, 11, and 18 days after treatment of HepG2_GFP/Luci cells co-cultured with M2 macrophages and T cells with 0.8 μg/mL TCE^{SR4}_CAR^{SR9} mRNA LNP: The indicated cells (center lower panels) received a second dose of TCE^{SR4}_CAR^{SR9} mRNA LNP at day 11 and were imaged 7 days later. The right panel shows luciferase activity (expressed as tumor burden relative to that of 18 days post-treatment) for the indicated treatments. The E/T for M2 was 1/64 and the E/T for T cells was 1/32.

FIG. 15A shows flow cytometry plots of Pan T cells used in the studies described in the above figures unstained (negative control) or stained with antibodies against CD45RO and CD3 (top right plot). CD45RO/CD3-positive cells were examined for CCR7 expression (bottom right plot). The CD45RO/CD3 positive cells are memory T cells (M.T.) and those cells included both central memory T cells (TCM) and effector memory T cells (TEM).

FIG. 15B shows flow cytometry plots of T cells recovered from the cultures of HepG2-GFP/Luci, M2, and Pan-T treated with TCE^{SR4}-CAR^{SR9} mRNA LNP described in FIG. 14. Abbreviations are shown in the legend to FIG. 15A.

FIG. 16 shows bar graphs summarizing the data from FIGS. 15A and 15B. Abbreviations are shown in the legend to FIG. 15A.

FIG. 17 shows bar graphs of luciferase activity (expressed as tumor burden relative to HepG2_GFP/Luci) of HepG2_GFP/Luci cells alone (HepG2), HepG2_GFP/Luci cells treated with T cells (Pan-T), and HepG2_GFP/Luci cells treated with MT cells generated in the TCE^{SR4}-CAR^{SR9} mRNA LNP treated co-culture study of HepG2_GFP/Luci cells+M2+T shown in FIGS. 12 and 13. SK_R1, SK_R2, and SK_R3 refer to rounds of treatment with MT serially collected from treated HepG2_GFP/Luci cells (R2 was treated with MT recovered from R1 and R3 was treated with MT recovered from R2).

FIG. 18 shows bar graphs of luciferase activity (expressed as tumor burden relative to HepG2_GFP/Luci) of HepG2_GFP/Luci (HepG2) and HepG2_GFP/Luci treated with T cells expanded from the corresponding co-cultures of HepG2_GFP/Luci/M2/TCE_SR4 and HepG2_GFP/Luci/M2/TCE_SR4-CAR_SR9 described in FIG. 11.

FIG. 19 is a diagram of the study design to examine in vivo pharmacological efficacy. NSG is NOD scid gamma mouse that is deficient in both B and T cells.

FIG. 20 is a plot of average tumor size versus days prior to or after the first of three injections close to or into the tumor of vehicle (control) or TCE^{SR4}_CAR^{SR9} mRNA LNP (TCE_CAR mRNA LNP) containing 4.5 μg each of TCE^{SR4} mRNA and CAR^{SR9} mRNA.

FIG. 21 is a plot of average body weight versus days following injection of TCE^{SR4}_CAR^{SR9} mRNA LNP or vehicle (control) as described in the legend to FIG. 20.

FIG. 22 is a plot of individual tumor size versus days prior to or after the first of three injections close to or into the tumor of vehicle (control) or TCE^{SR4}_CAR^{SR9} mRNA LNP as described in the legend to FIG. 20.

FIG. 23 shows flow cytometry analysis for the indicated markers of spleen cells collected from the cured cancer-free mice (5/7 mice showing no tumor growth in FIG. 22) in the left and center panels. The right panel is a bar graph showing average percentage of T cells expressing the indicated markers.

FIG. 24 is a plot of percent survival versus days post-injection of the mice described in the legend to FIG. 20.

FIG. 25 is a diagram of the study design to examine in vivo toxicology of the cancer treatment of the invention.

FIG. 26 is a plot of average tumor size versus days prior to or after the first of three injections near the tumor of vehicle (control) or TCE^{SR4}_CAR^{SR9} mRNA LNP (TCE_CAR mRNA LNP) containing 4.5 μg each of TCE^{SR4} mRNA and CAR^{SR9} mRNA.

FIG. 27 is a plot of average body weight versus days following injection of TCE^{SR4}_CAR^{SR9} mRNA LNP or vehicle (control) as described in the legend to FIG. 26.

FIG. 28A shows a bar graph of serum levels (units/L) of liver biomarker alanine aminotransferase (ALT) measured from mice with the indicated treatment. The horizontal dashed lines represent the normal range (NR) for the marker.

FIG. 28B shows a bar graph of serum levels (units/L) of liver biomarker aspartate aminotransferase (AST) measured from mice with the indicated treatment. The horizontal dashed lines are described in the legend to FIG. 28A.

FIG. 28C shows a bar graph of serum levels (mg/dL) of liver biomarker gamma-glutamyl transferase (GGT) measured from mice with the indicated treatment. The GGT levels were below the detection level of the assay (<7 mg/dL). The horizontal dashed lines are described in the legend to FIG. 28A.

FIG. 28D shows a bar graph of serum levels (mg/dL) of liver biomarker bilirubin measured from mice with the indicated treatment. The horizontal dashed lines are described in the legend to FIG. 28A.

FIG. 28E shows a bar graph of serum levels (units/L) of liver and kidney biomarker alkaline phosphatase (ALP) measured from mice with the indicated treatment. The horizontal dashed lines are described in the legend to FIG. 28A.

FIG. 28F shows a bar graph of serum levels (g/dL) of liver and kidney biomarker albumin measured from mice with the indicated treatment. The horizontal dashed lines are described in the legend to FIG. 28A.

FIG. 28G shows a bar graph of serum levels (mg/dL) of kidney biomarker blood urea nitrogen (BUN) measured from mice with the indicated treatment. The horizontal dashed lines are described in the legend to FIG. 28A.

FIG. 29A shows a bar graph of serum levels (units/L) of liver biomarker alanine aminotransferase (ALT) measured from the cured cancer-free mice (the 5/7 mice showing no tumor growth in FIG. 22 (TCE-CAR-LNP in situ)) and from NSG mice. The horizontal dashed lines are described in the legend to FIG. 28A.

FIG. 29B shows a bar graph of serum levels (units/L) of liver biomarker aspartate aminotransferase (AST) measured from the cancer-free mice and from NSG mice. The horizontal dashed lines are described in the legend to FIG. 28A.

FIG. 29C shows a bar graph of serum levels (mg/dL) of liver biomarker gamma-glutamyl transferase (GGT) measured from the cancer-free mice and from NSG mice. The GGT levels were below the detection level of the assay (<7 mg/dL). The horizontal dashed lines are described in the legend to FIG. 28A.

FIG. 29D shows a bar graph of serum levels (mg/dL) of liver biomarker bilirubin measured from the cancer-free mice and from NSG mice. The horizontal dashed lines are described in the legend to FIG. 28A.

FIG. 29E shows a bar graph of serum levels (units/L) of liver and kidney biomarker alkaline phosphatase (ALP) measured from the cancer-free mice and from NSG mice. The horizontal dashed lines are described in the legend to FIG. 28A.

FIG. 29F shows a bar graph of serum levels (g/dL) of liver and kidney biomarker albumin measured from the cancer-free mice and from NSG mice. The horizontal dashed lines are described in the legend to FIG. 28A.

FIG. 29G shows a bar graph of serum levels (mg/dL) of kidney biomarker blood urea nitrogen (BUN) measured from mice with the indicated treatment. The horizontal dashed lines are described in the legend to FIG. 28A.

### DETAILED DESCRIPTION

As mentioned in the SUMMARY section, a nanoparticle for treating HCC is disclosed. The nanoparticle includes a lipid phase and a core that contains one or more nucleic acids.

The lipid phase includes an ionizable lipid, cholesterol, a phospholipid and a polyethylene glycol-conjugated lipid (pegylated lipid).

Ionizable lipids refer to a lipid or lipid-like material capable of being positively charged and able to electrostatically bind nucleic acids. As used herein, a “cationic lipid” refers to a lipid or lipid-like material having a net positive charge. Cationic lipids or lipid-like materials bind negatively charged nucleic acid by electrostatic interaction. Generally, cationic lipids possess a lipophilic moiety, such as a sterol, an acyl chain, a diacyl, or more acyl chains, and the head group of the lipid typically carries the positive charge. Exemplary cationic lipids include one or more amine group(s) which bear the positive charge. Cationic lipids may encapsulate negatively charged RNA. In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “ionizable” lipids or lipid-like materials are comprised by the term “cationic lipid” or “cationic lipid-like material” unless contradicted by the circumstances. Examples of cationic lipids include, but are not limited to: ((4-hydroxybutyl)azanediyl)bis(hexane-6,1-diyl)bis(2-hexyldecanoate), 1,2-dioleoyl-3-trimethylammonium propane (DOTAP), N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB), 1,2-dioleoyl-3-dimethylammonium-propane (DODAP), 1,2-diacyloxy-3-dimethylammonium propanes, 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleoyloxypropyl-3-dimethyl-hydroxyethyl ammonium bromide (DORIE), 2,3-dioleoyloxy-N-[2 (spermine carboxamide)ethyl]-N,N-dimethyl-1-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-dilinoleoyloxy-N,N-dimethylpropylamine (DLinDAP), 1,2-N,N′-dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DMA), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy) propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio)-carbonyl)azanediyl) dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)oxy) heptadecanedioate (L319), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)-amino]ethyl-[2-[4-[2-[bis(2 hydroxydodecyl)amino]ethyl]piperazin-1-yl]ethyl]-amino]dodecan-2-ol (lipidoid 02-200); C12-200; or heptadecan-9-yl 8-((2-hydroxyethyl) (6-oxo-6-(undecyloxy) hexyl)amino) octanoate (SM-102). In some aspects, 1, 2, 3, 4, 5, or more of the foregoing cationic lipids may be excluded from the LNPs of the present disclosure.

Preferably, the ionizable lipid is (6Z,9Z,28Z,31Z)-heptatriaconta-6,9,28,31-tetraen-19-yl 4-(dimethylamino)butanoate (D-Lin-MC3-DMA), heptadecan-9-yl 8-((2-hydroxyethyl) (6-oxo-6-(undecyloxy) hexyl)amino) octanoate (SM102), 9Z,12Z-octadecadienoic acid, 3-[4,4-bis(octyloxy)-1-oxobutoxy]-2-[[[[3-(diethylamino)propoxy]carbonyl]oxy]methyl]propyl ester (LP01), 6-((2-hexyldecanoyl)oxy)-N-(6-((2-hexyldecanoyl)oxy) hexyl)-N-(4-hydroxybutyl) hexan-1-aminium (ALC-0315), and analogs thereof. Analogs of the ionizable lipids include modifications of one or more of the head, chemical linkage, bridge chain, and tail portion.

The chemical nature changes of the ionizable lipid's head amine group, e.g., substituting different amine groups such as tertiary amines, secondary amines, or cyclic amines, can affect its pKa value, which impacts how well the head interacts with nucleic acids inside an acidic endosome.

The ionizable lipid's chemical linkage (bridge chain) connects the head group to the tail. Its alteration, e.g., introducing ester, amide, ether, or disulfide linkages, can influence the degradation rate and stability of the ionizable lipid. This alteration can also affect endosomal escape.

The modification of the ionizable lipid tail includes using different fatty acid chains with variations of chain lengths, unsaturation degree, and/or branching patterns. It can impact the ionizable lipid overall hydrophobicity and membrane-disrupting capabilities.

The ionizable lipid typically constitutes by mole percentage 5-95% (e.g., 37% to 47%, 10-90%, 15-85%, 20-80%, 25-70%, 30-60%, 35-50%, 38%, 39%, 40%, 41%, 42%, 43%, 44%, 45%, and 46%). Further a molar ratio of the ionizable lipid to the one or more nucleic acids is preferably in the range of 2:1 to 6:1 (e.g., 2.5:1 to 5.9:1, 3:1 to 5.8:1, and 3.3:1 to 5.6:1).

The nanoparticle further contains cholesterol or cholesterol derivative. Examples of cholesterol derivatives include, but are not limited to, cholestanol, 5,6-epoxy cholestanol, cholestanone, cholestenone, coprostanol, cholesteryl-2′-hydroxyethyl ether, cholesteryl-4′-hydroxybutyl ether, 24-ethyl cholesterol, 24-methyl cholesterol, cholenic Acid, 3-hydroxy-5-cholestenoic Acid, cholesteryl palmitate, cholesteryl arachidonate, cholesteryl arachidate, cholesteryl myristate, cholesteryl palmitoleate, cholesteryl lignocerate, cholesteryl oleate, cholesteryl stearate, cholesteryl erucate, cholesterol α-linolenate, cholesteryl linoleate, cholesteryl homo-γ-linolenate, 4-hydroxy cholesterol, 6-hydroxy cholesterol, 7-hydroxy cholesterol, 19-hydroxy cholesterol, 20-hydroxy cholesterol, 22-hydroxy cholesterol, 24-hydroxy cholesterol, 25-hydroxy cholesterol, 27-hydroxy cholesterol, 27-alkyne cholesterol, 7-keto cholesterol, 7-dehydro cholesterol, 8-dehydro cholesterol, 24-dehydro cholesterol, 5α-hydroxy-6-keto cholesterol, 20,22-dihydroxy cholesterol, 7,25-dihydroxy cholesterol, 7,27-dihydroxy cholesterol, 7-keto-25-hydroxy cholesterol, fucosterol, phytosterol, cholesteryl 11,14-eicosadienoate, dimethyl hydroxyethyl aminopropane carbamoyl cholesterol iodide and mixtures thereof. The cholesterol derivative may comprise a sugar moiety and/or amino acids. In an embodiment the amino acids are selected from serine, threonine, lysine, histidine, arginine or their derivatives.

The nanoparticle includes the cholesterol or cholesterol derivative in an amount typically ranging by mole percentage 1% to 90% (e.g., 42% to 50%, 5% to 85%, 10% to 80%, 15% to 75%, 20% to 70%, 25% to 65%, 30% to 60%, 35% to 55%, 43%, 44%, 45%, 46%, 47%, 48%, and 49%).

Exemplary phospholipids include phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), 1,2-diundecanoyl-sn-glycero-phosphocholine (DUPC), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), 1-phytanoyl-phosphatidylethanolamine (DpyPE), 16-O-monomethyl PE, 16-O-dimethyl PE, 18-1-trans PE, 1-stearoyl-2-oleoylphosphatidyethanolamine (SOPE), 1,2-dielaidoyl-sn-glycero-3-phosphoethanolamine (transDOPE), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some aspects, 1, 2, 3, 4, 5, or more of the foregoing neutral lipids may be excluded from the LNPs of the present disclosure. Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from or from about 2:1 to about 8:1, or from or from about 10:0 to about 1:9, about 4:1 to about 1:2, or about 3:1 to about 1:1. In another aspect, the molar ratio of the cationic lipid to cholesterol ranges from or from about 2:1 to 1:1. In a further aspect, the molar ratio of the cationic lipid to the pegylated lipid ranges from or from about 100:1 to about 10:1 or from about 100:1 to about 25.1. In some aspects, the non-cationic lipid, e.g., neutral lipid (e.g., one or more phospholipids and/or cholesterol), may comprise from or from about 0 mol % to about 90 mol %, from or from about 0 mol % to about 80 mol %, from or from about 0 mol % to about 70 mol %, from or from about 0 mol % to about 60 mol %, or from or from about 0 mol % to about 50 mol %, of the total lipid present in the particle. In some aspects, the non-cationic lipid, e.g., neutral lipid (e.g., one or more phospholipids and/or cholesterol), may or may not be at least, at most, exactly, or between (inclusive or exclusive) of 0 mol %, 10 mol %, 20 mol %, 30 mol %, 40 mol %, 50 mol %, 60 mol %, 70 mol %, 80 mol %, or 90 mol % of the total lipid present in the particle. In some aspects, the phospholipid is in the range, by molar percentage, of 1% to 30% (e.g., 7% to 12%, 2% to 25%, 3% to 20%, 4% to 18%, 5% to 15%, 8%, 9%, 10%, and 11%).

Preferred phospholipid include dilinoleoylphosphatidylcholine (DLPC), dipalmitoylphosphatidylcholine (DPPC), distearoylphosphatidic acid (DSPA), dipalmitoylphosphatidic acid (DPPA), distearoylphosphatidylcholine (DSPC), dimyristoylphosphatidylethanolamine (DMPE), diheptanoylphosphatidylcholine (DHPC), dimyristoylphosphatidylcholine (DMPC), stearoyl-palmitoylphosphatidylcholine (SPPC), and diarachidoylphosphatidylcholine (DAPC).

Further, the pegylated lipids include polyethylene glycol (PEG)-lipid conjugates, such as PEG coupled to lipids (for example, DMG-PEG 2000), PEG coupled to phospholipids (for example, phosphatidylethanolamine (PEG-PE)), PEG conjugated to cholesterol or a derivative thereof, and mixtures thereof. In certain instances, PEG may be optionally substituted by alkyl, alkoxy, acyl, or aryl group.

Useful PEGs in the PEG-lipid conjugates are classified by their molecular weights; for example, PEG 2000 has an average molecular weight of about 2,000 Daltons, and PEG 5000 has an average molecular weight of about 5,000 Daltons. PEGs are commercially available from suppliers such as Avanti Polar Lipids. The PEG moiety of the PEG-lipid conjugates described herein may comprise a number-average molecular weight ranging from 550 Daltons to 20,000 Daltons (e.g., 600 Daltons to 15,000 Daltons, 800 Daltons to 10,000 Daltons, 900 Daltons to 8,000 Daltons, 1,000 Daltons to 6,000 Daltons, 1,200 Daltons to 5,000 Daltons, 1,300 Daltons to 4,000 Daltons, 1,400 Daltons to 3,500 Daltons, 1,500 Daltons to 3,000 Daltons, 1,600 Daltons, 1,800 Daltons, 1,900 Daltons, 2,000 Daltons, 2,100 Daltons, 2,200 Daltons, and 2,500 Daltons).

Suitable lipids for conjugating with PEG include phosphatidylethanolamines, diacylglycerols, phosphatidic acids, ceramides, dialkylamines, dialkylglycerols, 1,2-diacyloxypropan-3-amines, and mixtures thereof. These lipids have a variety of acyl chain groups of varying chain lengths and degrees of saturation. They are commercially available or can be isolated or synthesized using conventional techniques. The lipids can comprise saturated or unsaturated fatty acids with carbon chain lengths in the range of C₁₀ to C₂₀. They can contain mono- or polyunsaturated fatty acids and mixtures of saturated and unsaturated fatty acids. The lipids contemplated include, but are not limited to, dimyristoylglycerol (DMG), dimyristoylphosphatidylethanolamine (DMPE), dipalmitoyl-phosphatidylethanol amine (DPPE), dioleoylphosphatidylethanolamine (DOPE), and distearoylphosphatidylethanolamine (DSPE).

Suitable pegylated lipids are known in the art, e.g., WO2025238563A1. Examples include distearoylphosphatidylethanolamine-polyethylene glycol 2000 (DSPE-PEG 2000), dimyristoyl-PEG 2000 (DMG-PEG 2000), DSPE-PEG 2000-Mannose, DMG-PEG 2000-Mannose, PEG-c-DOMG, PEG-DLPE, PEG-DMPE, PEG-DPPC, PEG-ceramide conjugates (e.g., PEG-CerCI4 or PEG-CerC20), and PEG-c-DOMG. The pegylated lipid constitutes by molar percentage from 0.1% to 30% (e.g., 1.5% to 5.5%, 0.2% to 25%, 0.3% to 20%, 0.4% to 15%, 0.5% to 10%, 0.8% to 8%, 1% to 7%, 2%, 2.5%, 3%, 3.5%, 4%, 4.5%, and 5%).

Preferred pegylated lipids include distearoylphosphatidylethanolamine-polyethylene glycol 2000 (DSPE-PEG 2000), dimyristoyl-PEG 2000 (DMG-PEG 2000), DSPE-PEG 2000-Mannose, and DMG-PEG 2000-Mannose.

In certain embodiments, the lipid phase includes at least two pegylated lipids selected from DSPE-PEG 2000, DMG-PEG-2000, DSPE-PEG 2000-Mannose, and DMG-PEG-2000-Mannose.

The mole percentage of the ionizable lipid, the cholesterol, the phospholipid, and the pegylated lipid in the nanoparticle is 37% to 47%, 42% to 50%, 7% to 12%, and 1.5% to 5.5%, respectively.

As also mentioned above, the nanoparticle includes a core containing one or more nucleic acid. The core can include a buffer solution. The buffer can be at a pH of 4.5 to 6.5 at a concentration of 25 mM to 300 mM. Exemplary buffers include sodium citrate buffer and sodium acetate buffer.

The nanoparticle has a molar ratio of the ionizable lipid to the one or more nucleic acids ranging from 2:1 to 6:1, preferably 3:1 to 6:1, more preferably 3.3:1 to 5.6:1.

The one or more nucleic acid in the nanoparticle of the invention can encode a chimeric antigen receptor (CAR), a T-cell engager (TCE), and/or a therapeutic gene. In a particular embodiment, the nucleic acid is an mRNA molecule encoding the CAR, TCE, or therapeutic gene. In a specific embodiment, the CAR, TCE, and therapeutic gene are encoded by separate mRNA molecules. Alternatively, a single mRNA molecule can encode the CAR, TCE, and therapeutic gene. In a preferred embodiment, the nanoparticle includes two individual mRNA molecules, one encoding the CAR and the other encoding the TCE.

Not to be bound by theory, the combination of the CAR and the TCE will expand the number of tumor-associated antigen (TAA) targets to overcome solid tumor associated genetic heterogeneity and therapy-induced targets loss. In addition, the activation and mobilization of the bystander T cells in the TME by the TCE will cross talk with macrophage to initiate neoantigen cancer vaccine in the treated solid tumor TME. It will alleviate TME immunosuppression and result in long term therapeutic efficacy.

The CAR includes a binding domain that specifically recognizes an HCC TAA. The TAA can be, but is not limited to, glypican-3 (GPC-3), alpha-fetoprotein (AFP), HSP70, AFP-L3, des-gamma-carboxy prothrombin (DCP), NY-ESO-1, EpCAM, CK19, EGFR, EGFRVIII, annexin A2, MAGE-C1, and MAGE-C2.

In some embodiments, the CAR also includes a CD8a signal peptide, a CD8a hinge, a CD28 transmembrane domain, a CD32 signaling domain, and/or a 4-1BB costimulatory domain, and/or a TRL4-TIR domain, or combinations of these domains.

The CAR can have (include or consist of) the amino acid sequence of any one of SEQ ID NOs 6 to 9 or amino acid sequences having 75% to 99% similarity (75%, 80%, 85%, 90%, 95%, 99%) to SEQ ID NOs 6 to 9. Nucleic acids encoding these CAR are also within the scope of the invention.

The TCE includes a binding domain that specifically binds to an HCC TAA and a binding domain that specifically binds to T cells via CD3 or CD137 (4-1BB). In certain embodiments, the TCE includes two HCC TAA binding domains and a T cell binding domain.

The HCC TAA binding domain of the TCE can be, but is not limited to, glypican-3 (GPC-3), alpha-fetoprotein (AFP), HSP70, PD-L1, AFP-L3, des-gamma-carboxy prothrombin (DCP), NY-ESO-1, EpCAM, CK19, EGFR, EGFRVIII, annexin A2, MAGE-C1, and MAGE-C2.

The TCE can have (include or consist of) the amino acid sequence of any one of SEQ ID NOs 1 to 5 or amino acid sequences having 75% to 99% similarity (75%, 80%, 85%, 90%, 95%, 99%) to SEQ ID NOs 1 to 5. Nucleic acids encoding these TCE are also within the scope of the invention.

In a particular nanoparticle, the mRNAs encode (i) a TCE having an HCC TAA binding domain and (ii) a CAR having the same HCC TAA binding domain as the TCE. Alternatively, the HCC TAA binding domain of the TCE is distinct from the HCC TAA binding domain of the CAR. Further, in an embodiment where the TCE includes two HCC TAA binding domains, the two binding domains can bind to distinct HCC TAAs or to the same HCC TAA.

As mentioned above, the mRNA can also encode a therapeutic gene. The therapeutic gene provides additional anti-cancer efficacy to the therapy. The therapeutic gene can be, e.g., an anti-VEGF antibody, an anti-VEGFR antibody, IL-2, IL-2 muteins, TGFβ-trap, TGFβ receptor muteins, anti-PD-1 antibody, anti-PD-L1 antibody, and anti-CD47 antibody.

The following method can be used to prepare the nanoparticle of the invention. The lipid phase components are mixed together at room temperature in a solvent, e.g., absolute ethanol, in the molar percentages set forth, supra. Separately, the one or more nucleic acid is formulated, also at room temperature, in a solution containing a buffer, e.g., sodium acetate buffer, to stabilize the nucleic acid. The lipid phase and solution are then mixed using an automated mixing instrument in a 1:1 to 1:5 v/v ratio of lipid phase to solution with a flow rate of 2 to 100 mL per minute at room temperature.

Also within the scope of the invention is a therapeutic composition that contains a plurality of the nanoparticle described above, together with a pharmaceutically acceptable excipient, e.g., solvents, salts, and sugars/cryoprotectants.

Another method is provided for converting HCC tumor-resident M2 macrophages into M1 macrophages by contacting the M2 macrophages with a plurality of the above-described nanoparticle. The contacting step is carried out in vivo by delivering the composition into an HCC tumor that contains M2 macrophages. Delivering the composition into an HCC tumor can be accomplished by a route that includes, but is not limited to, hepatic artery infusion, intra-tumor injection, and IV infusion.

As summarized above, also disclosed is a method for treating HCC in a subject by administering a nanoparticle of the invention to a subject suffering from HCC. The nanoparticle includes one or more nucleic acids that encode a CAR, a TCE, and, optionally, a therapeutic gene. The CAR, TCE, and therapeutic gene are those described above. An exemplary nanoparticle includes in its core an mRNA encoding a CAR that specifically binds to GPC-3, an mRNA encoding a TCE that specifically binds to an HCC TAA and to CD3, and an mRNA encoding an anti-VEGF antibody.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications, including patent documents, cited herein are incorporated by reference in their entirety.

### EXAMPLES

### Example 1: Identification of HCC Lead CAR mRNA LNP with High Potency at a Low E/T Ratio

Tumor-associated macrophages (TAM; M2) are a dominant population of immune cells in the HCC TME and key contributors to TME immune suppression. Targeting TAM and flipping their immune suppression switch is an ideal therapeutic approach. A series of anti-glypican 3 (GPC3) CARs were constructed with differentiated intracellular stimulation/activation domains (constructs CAR_SR6-SR9; see Table 1 below).

TABLE 1
CAR and TCE specificities and corresponding SEQ ID NOs

| Construct | Specificities | SEQ ID NO |
|---|---|---|
| TCESR1 | anti-EGFR/EpCAM/CD3 | SEQ ID NO: 1 |
| TCESR2 | anti-EGFR/EpCAM/CD3 | SEQ ID NO: 2 |
| TCESR3 | anti-EGFR/EpCAM/CD3 | SEQ ID NO: 3 |
| TCESR4 | anti-EGFR/EpCAM/CD3 | SEQ ID NO: 4 |
| TCESR5 | anti-EpCAM/CD3 | SEQ ID NO: 5 |
| CARSR6 | GPC3 | SEQ ID NO: 6 |
| CARSR7 | GPC3 | SEQ ID NO: 7 |
| CARSR8 | GPC3 | SEQ ID NO: 8 |
| CARSR9 | GPC3 | SEQ ID NO: 9 |
| mRNA IVT Plasmid | N/A | SEQ ID NO: 10 |

IVT was performed with pseudo-uridine and a capping reagent. LNP lipid components are described above. Exemplary LNPs could selectively transfect M2 macrophages with high efficiency. See Table 2 below.

TABLE 2
LNP formulations

| | | Molar Ratio of | | |
|---|---|---|---|---|
| mRNA | | Ionizable Lipid | % of | Expression |
| LNP | | to Nucleic | Transfected | Efficacy |
| Type | Pegylated Lipid | Acids | Cells | (+) |
| LNP_A | DMG-PEG-2000- | 4.5:1 | 25 | + |
| | Mannose | | | |
| LNP_B | DMG-PEG-2000 | 4.5:1 | 95 | ++++ |
| LNP_C | DSPE-PEG 2000 | 4.5:1 | 11 | + |
| LNP_D | DSPE-PEG 2000- | 4.5:1 | 12 | + |
| | Mannose | | | |
| LNP_E | 75% of DMG- | 3.5:1 | 95 | +++ |
| | PEG-2000 | | | |
| | 25% of DSPE- | | | |
| | PEG 2000- | | | |
| | Mannose | | | |

The mRNA LNPs were produced with the iNanno L as directed by the manufacturer (MicroNano) using its single molecule detector (SMD) card at room temperature. The mRNA LNPs were stored in 10% (w/v) sucrose at −80° C. The LNP particle size, surface charge and concentration were analyzed using Zeta Sizer Red.

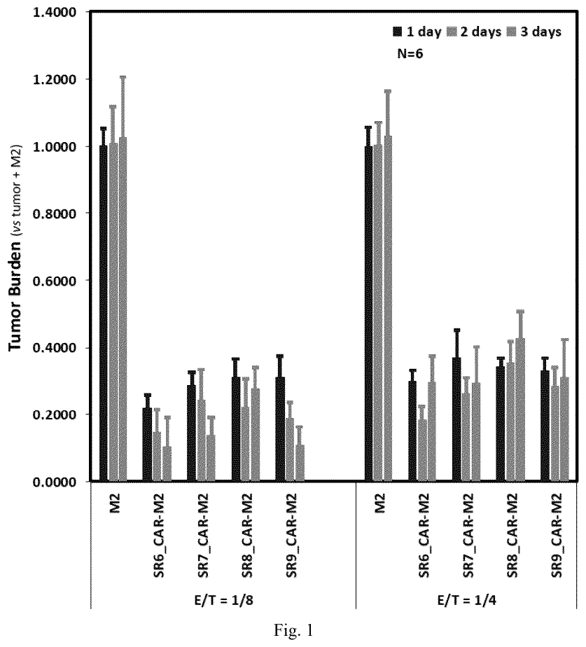

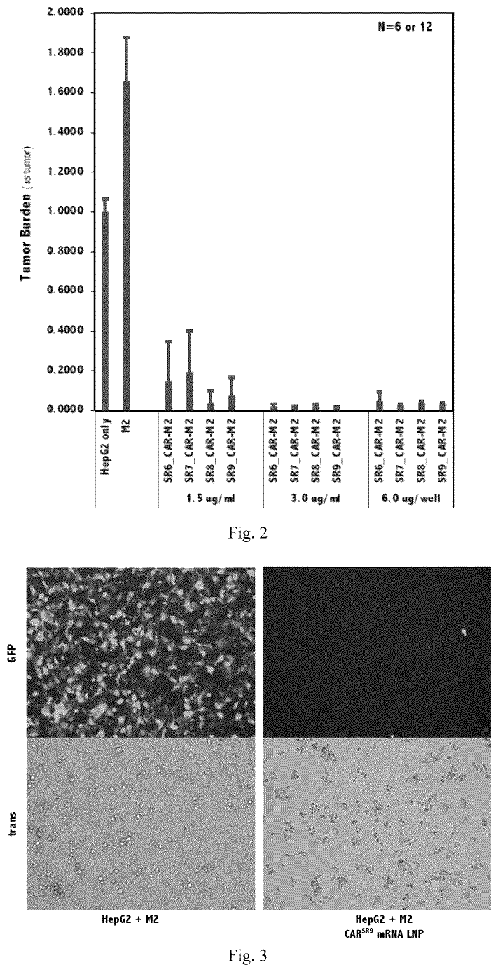

HepG2 HCC cells stably expressing both GFP and luciferase (HepG2_GFP/Luci) were co-cultured with M2 macrophages at different effector (M2) to target (HCC) ratios and treated with CAR mRNA LNPs. Luciferase activities in the cocultures were measured at different times after CAR mRNA LNP treatment and compared to cocultures not treated with the CAR mRNA LNPs. The results are shown in FIGS. 1 and 2. CAR_SR9 was selected as the most potent at the lowest E/T ratio of 1/8.

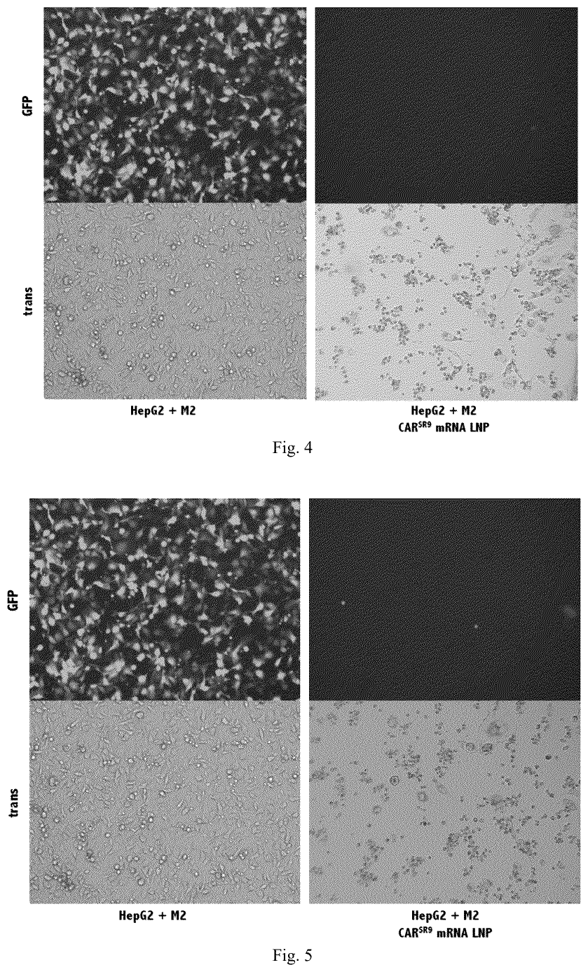

The effectiveness of CAR_SR9 was confirmed by directly observing GFP expression in the HCC cells cocultured with M2 and treated with the CAR mRNA LNP. The results are shown in FIGS. 3-5.

### Example 2: Identification of HCC Lead TCE mRNA LNP with High Potency at a Low E/T Ratio

To further harness the therapeutic potentials of LNPs that selectively target human M2 macrophages, a series of anti-EGFR and/or -EpCAM T Cell Engager (TCE) constructs were made (constructs TCE_SR1-SR5; see Table 1 above) and used as IVT templates to produce mRNA, which was encapsulated into LNPs. The resulting TCE mRNA LNP were used to transfect M2 macrophages that subsequently secreted the TCEs.

Supernatants from transfected cultured M2 macrophages were used in a T cell activation assay that employed cocultures of HepG2 cells and Jurkat T cells stably transfected with an NFAT-luciferase construct. HCC cancer cells at a concentration of 20,000 cells/well were plated in 96-well plates, and on the next day, Jurkat (NFAT-Luciferase) reporter cells as well as TCEs collected from mRNA LNP treated M2 macrophage supernatants were added at an E/T ratio (Jurkat/HCC) of ½ for 10, 12, or 24 hrs. Luciferase activity was then assessed using ONE-Step™ Luciferase assay system and luminescence was measured in a microplate reader.

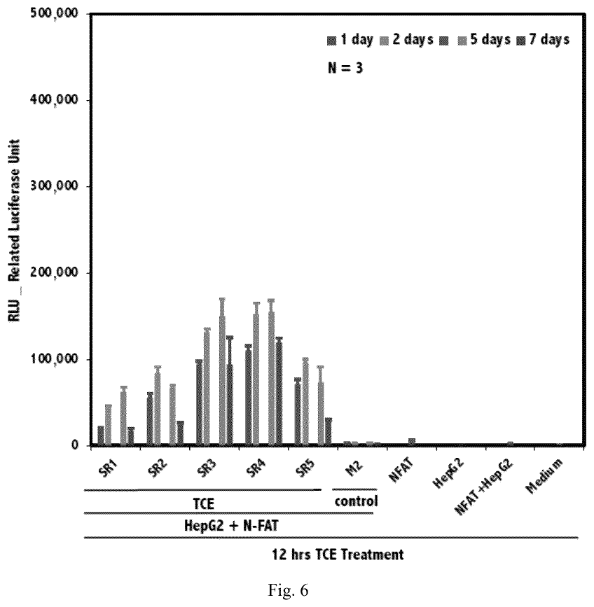

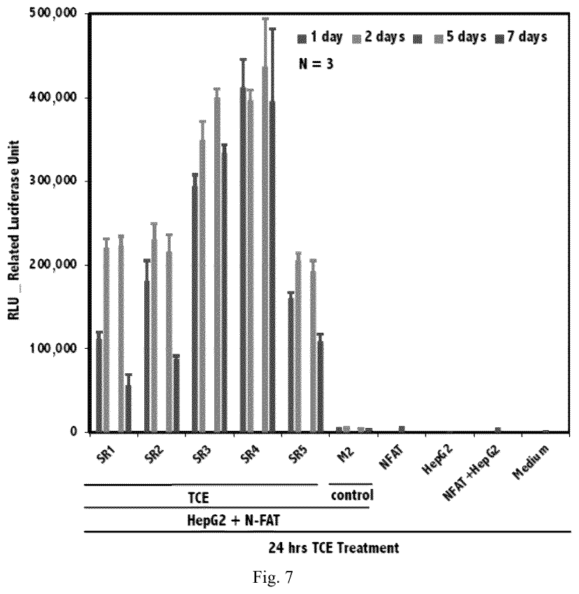

The results are shown in FIGS. 6 and 7. The TCE secreted into transfected M2 culture supernatants activated the Jurkat T cells in a manner dependent on the presence of HepG2 cells.

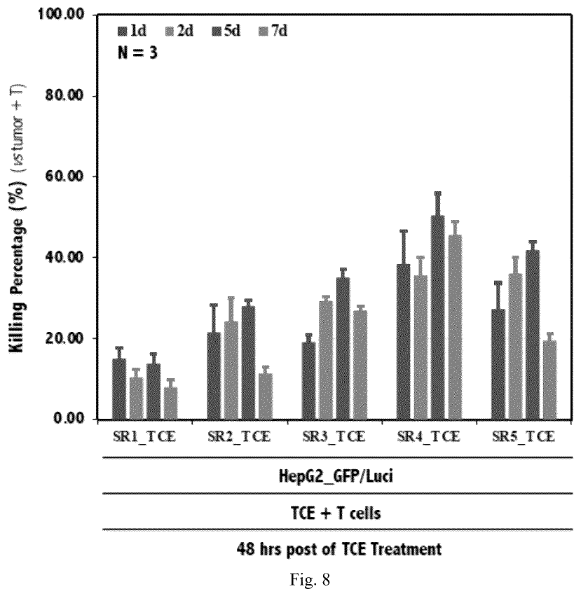

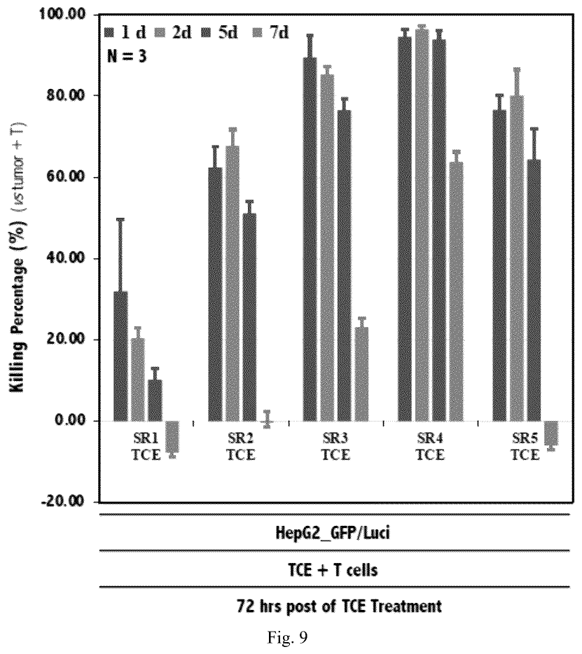

The ability of the secreted TCE to activate T cells to kill HCC cells was tested by treating cultured HepG2_GFP/Luci cells with Pan-T cells and the secreted TCE. The results are shown in FIGS. 8 and 9. All of the TCE induced T cells to kill the HCC cells to a degree, the most potent being TCE_SR4.

### Example 3: Identification of HCC Lead Composition of TCE_CAR mRNA LNP with High Potency Even at an Extremely Low E/T Ratio

After identification of lead CAR and TCE for treating HCC, both CAR and TCE mRNAs were simultaneously encapsulated into LNP targeting M2 macrophages. The resulting TCE_CAR mRNA LNP (more specifically TCE^{SR4}_CAR^{SR9} mRNA LNP) was tested for its ability to kill HCC cells (HepG2_GFP/Luci) cocultured with M2 macrophages and T cells. The results are shown in FIGS. 10-13.

Treatment of HCC/M2/T cell cocultures with TCE_CAR LNP resulted in significantly more killing of the HCC cells, as compared to CAR mRNA LNP, despite the fact that the TCE_CAR LNP carries half the amount of mRNA for the CAR compared to the CAR mRNA LNP. See FIG. 10.

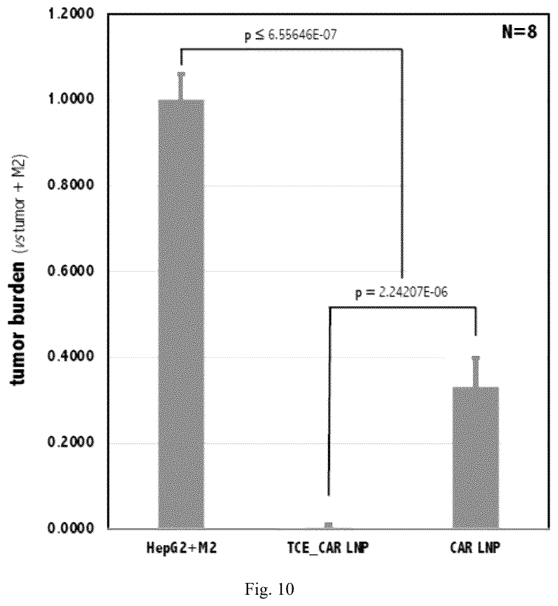

A similar study showed that the potency of the TCE_CAR LNP for killing HCC was significantly better than the TCE mRNA LNP. See FIG. 11.

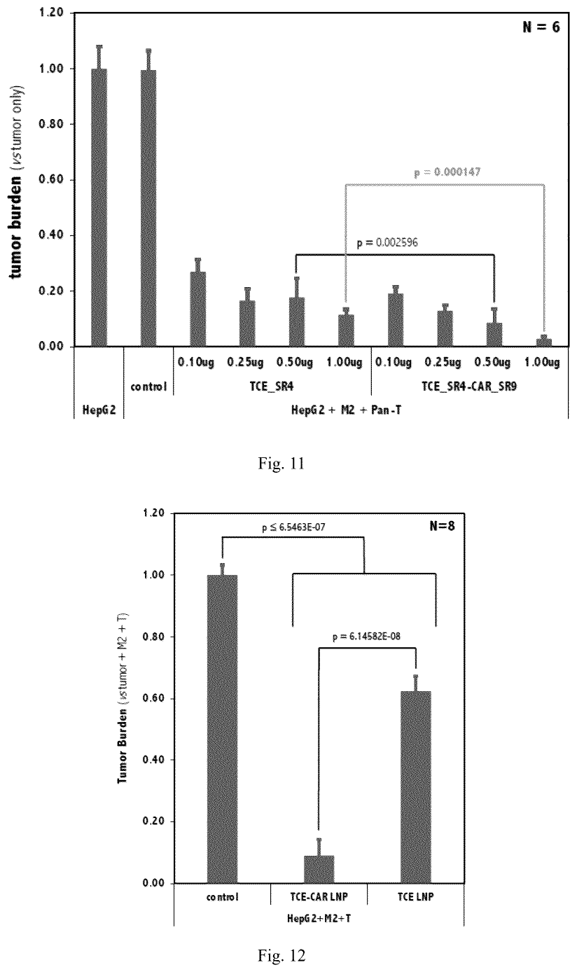

Further studies revealed that, even at very low E/T ratios (E/T M2=1/64; E/T T=1/32), the TCE_CAR mRNA LNP killed significantly more HCC cells compared to the TCE mRNA LNP. See FIG. 12.

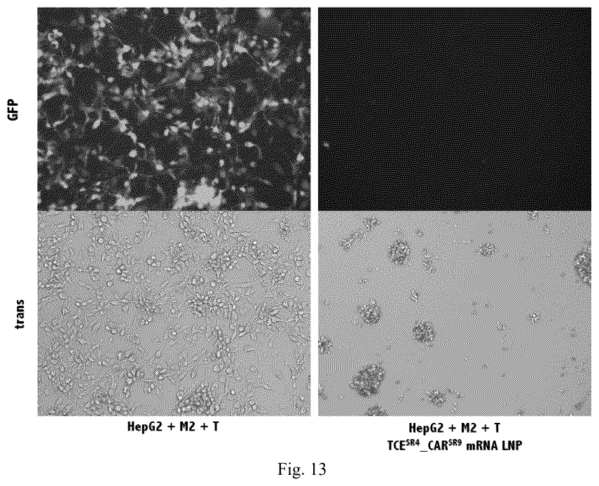

The effectiveness of the TCE_CAR mRNA LNP was confirmed by directly observing GFP expression in the HCC cells cocultured with M2 and T cells and treated with TCE^{SR4}_CAR^{SR9} mRNA LNP. See FIG. 13.

### Example 4: Repeat Dosing of the Lead TCE_CAR mRNA LNP has a Robust Killing Potency to Human HCC Cancer Cells

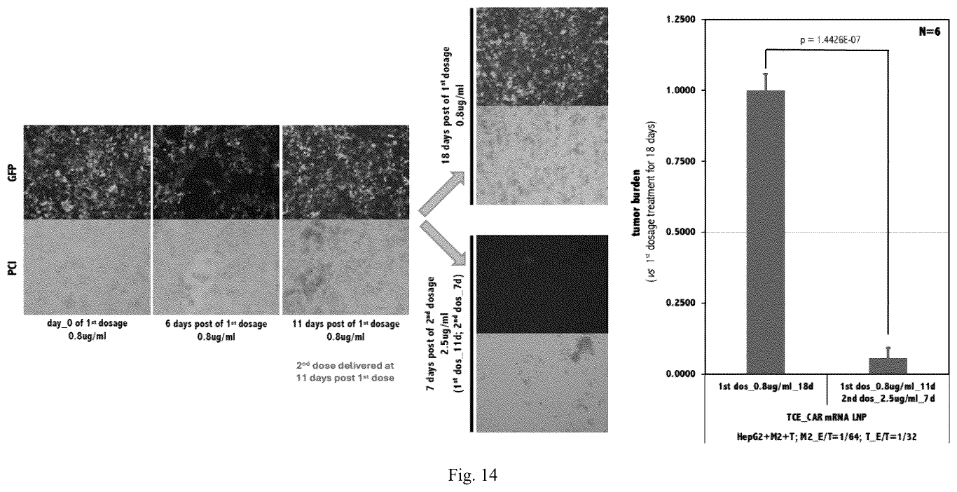

An in vitro co-culture study was performed to directly address the question whether recurrent HCC tumor can be eradicated by repeated dosing of TCE_CAR mRNA LNP. The results are shown in FIG. 14. HepG2_GFP/Luci cells were co-cultured with M2 macrophages and T cells and treated with 0.8 μg/mL TCE^{SR4}_CAR^{SR9} mRNA LNP. Significant killing of the HCC cells was seen after 6 days, but HCC cell numbers returned to pre-treatment levels by 11 days post-treatment. See FIG. 14, left panel. On day 11, some of the previously treated HCC cells received a second dose of TCE^{SR4}_CAR^{SR9} mRNA LNP and others were left untreated. The results showed that nearly all of the HCC cells receiving the second dose were killed within 7 days (18 days after the initial treatment) as compared to cells treated only once. See FIG. 14, center and right panels. This repeat dose study indicated that TCE^{SR4}_CAR^{SR9} mRNA LNP had a robust killing activity against recurrent human HCC cancer cells.

### Example 5: Generation of Memory T Cells (Cancer Vaccine) Via the Interplay of CAR and TCE

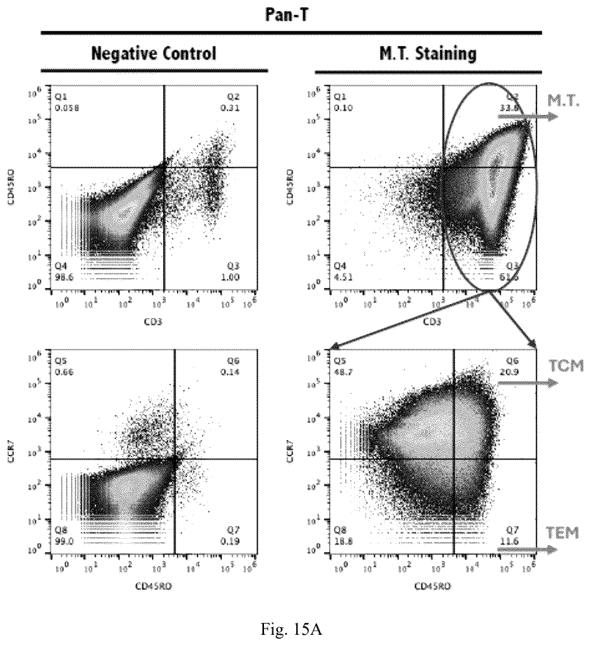

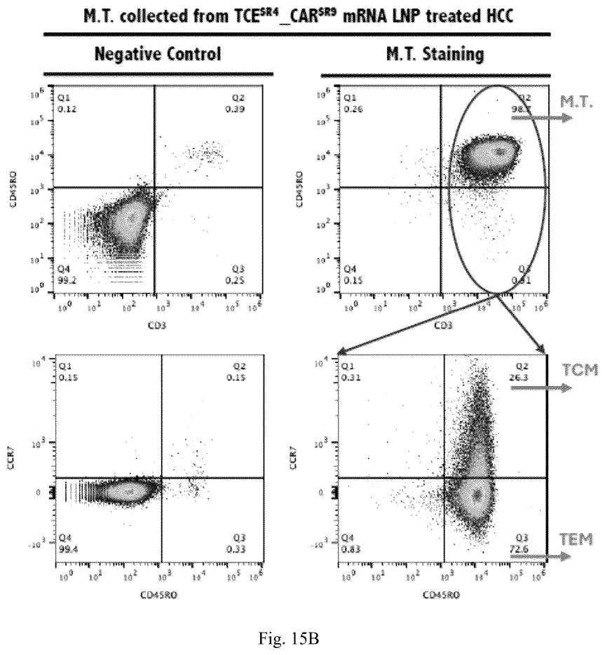

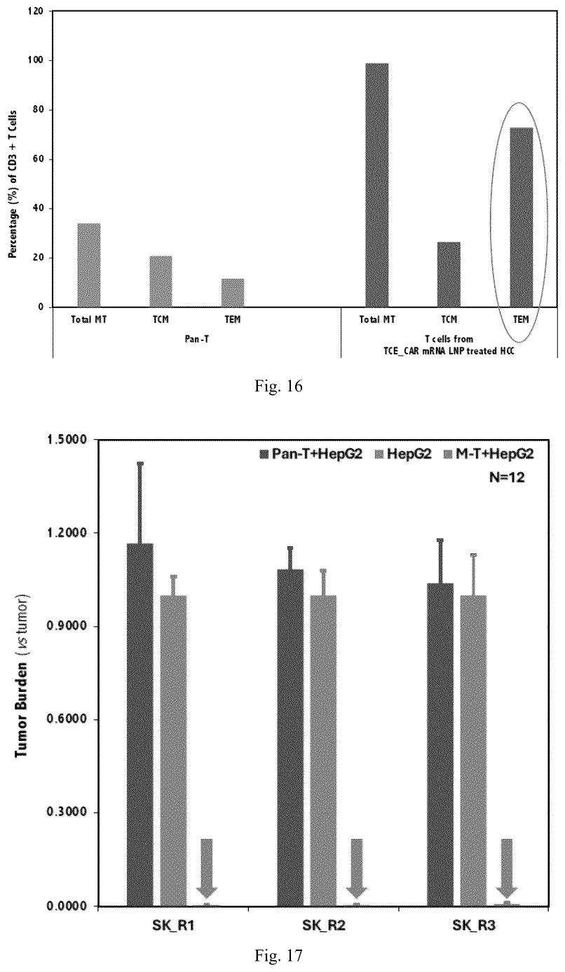

The effect of the above treatments on T cell populations was examined by flow cytometry analysis. As shown in FIGS. 15A and 16, about 34% of Pan-T cells were memory T cells (MT), with 21% being central memory T cells (TCM) and 12% being effector memory T cells (TEM).

The T cells that were expanded during the HCC cell-killing study described in Example 3 and shown in FIG. 13 (TCE^{SR4}_CAR^{SR9} mRNA LNP treated HCC cancer cells co-cultured with M2 macrophages and T cells) surprisingly were 99% MT, with 26% TCM and 73% TEM. See FIGS. 15B and 16.

The MT cells recovered from the HCC cell-killing study effectively killed HCC cells in the absence of added M2 cells. See FIG. 17, SK_R1. T cells recovered from SK_R1 were similarly effective at killing HCC cells. See FIG. 17, SK_R2. Another serial round of killing showed again that T cells from the second serial killing round were effective for killing HCC cells. See FIG. 17, SK_R3.

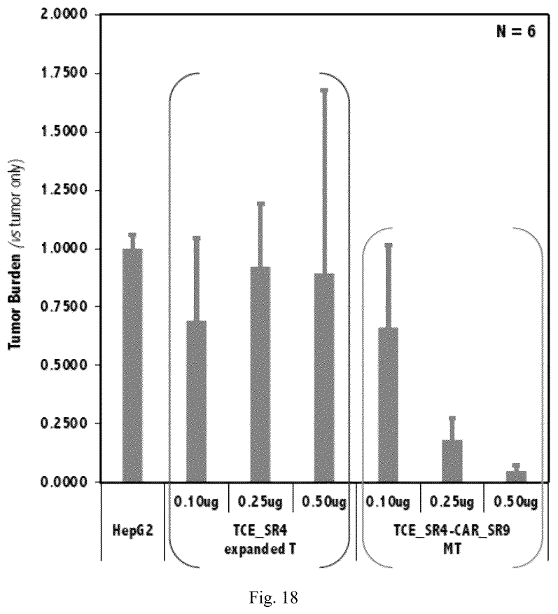

T cells expanded from the coculture studies described in Example 3 and shown in FIG. 13 were tested for their ability to kill HCC cells. The results are shown in FIG. 18. Surprisingly, T cells from cocultures of HCC/M2/T cells treated with TCE^{SR4} mRNA LNP were relatively ineffective at killing HCC cells, while T cells from cocultures of HCC/M2/T cells treated with TCE^{SR4}_CAR^{SR9} mRNA LNP had significant ability to kill the HCC cells. This result indicates that the cancer vaccine response was generated via the interplay of CAR and TCE.

### Example 6: The Lead TCE_CAR mRNA LNP has a Significant In Vivo Therapeutic Efficacy Against HCC Tumor

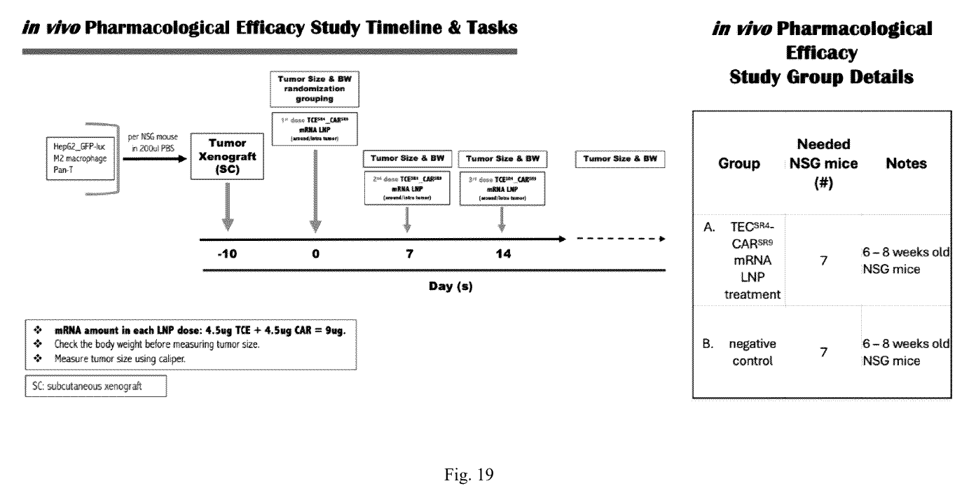

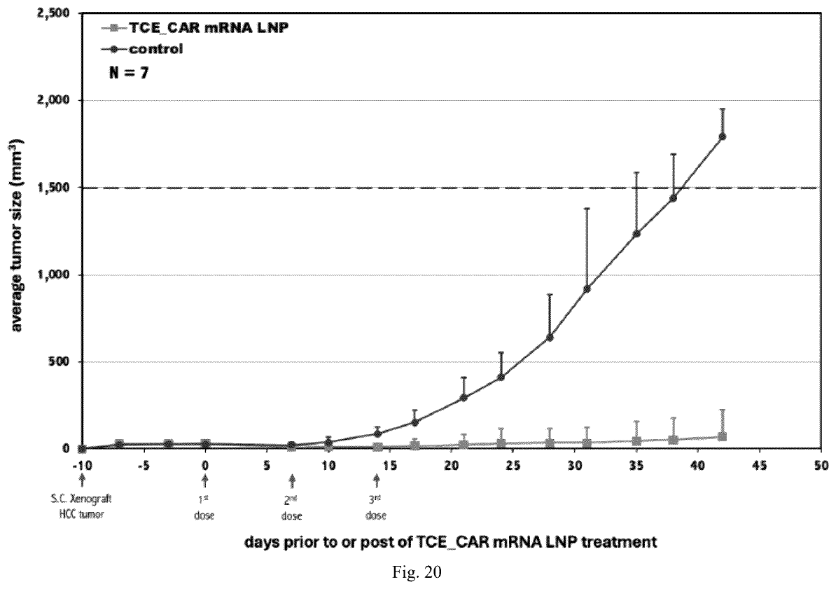

In vivo efficacy studies were performed in a murine model for HCC according to the design shown in FIG. 19. Briefly, HCC tumors were established in NSG mice by subcutaneous injection of 7.5×10⁶ HepG2_GFP/Luci cells, 2.5×10⁶ human M2 macrophages, and 2.5×10⁶ human T cells. Ten days later (Day 0) the mice were randomized to treatment group and control group. The treatment group mice were injected into or near the established tumors with an amount of TCE^{SR4}-CAR^{SR9} mRNA LNP containing 4.5 μg of each mRNA for a total of 9 μg. Control animals were injected with vehicle. Injections were repeated on day 7 and day 14, and tumor size measured on days −7, −3, 0, 7, 10, 14, 17, 21, 24, 28, 31, 35, 38, and 42. The tumor size measurements are shown in FIG. 20. Treatment with TCE^{SR4}-CAR^{SR9} mRNA LNP significantly reduced tumor size by day 14. See Table 3 below.

TABLE 3
Significant differences in tumor size between
TCE^{SR4}-CAR^{SR9} mRNA LNP treated and controls

| Days | 0 | 7 | 10 | 14 | 17 | 21 |
|---|---|---|---|---|---|---|
| p | 0.06322799 | 0.13246355 | 0.06077777 | 0.0069429 | 0.00343986 | 0.00137466 |

| Days | 24 | 28 | 31 | 35 | 38 | 42 |
|---|---|---|---|---|---|---|
| p | 0.00080167 | 0.0054513 | 0.00107546 | 7.0924E−05 | 6.8163E−06 | 6.3335E−07 |

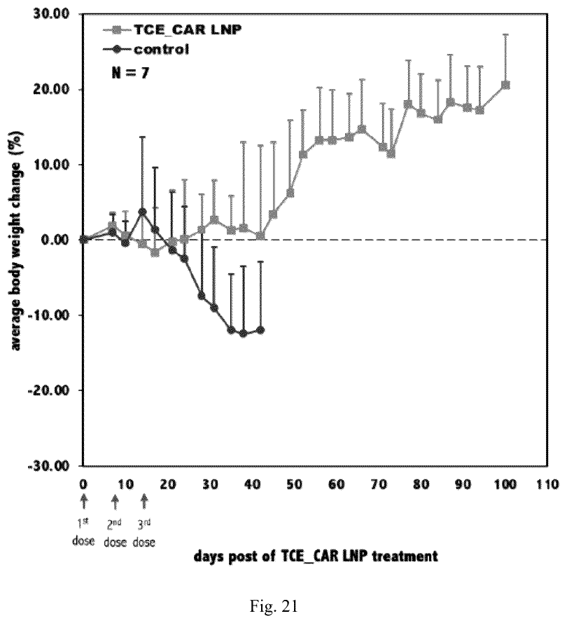

Average body weight of treated and control mice were also measured starting on day 0. The results are shown in FIG. 21. Control mice lost a significant amount of body weight over the course of the study, while treated mice maintained body weight and then showed an increase after day 42. The increase in weight was expected for the age of the mice.

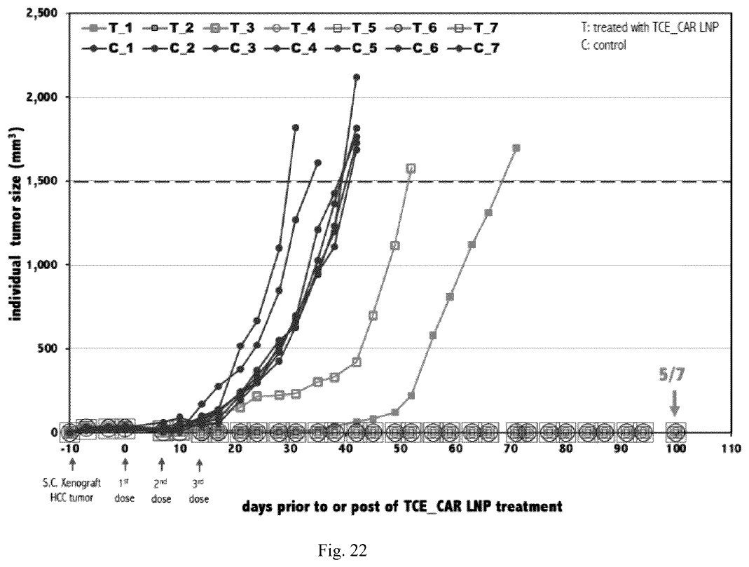

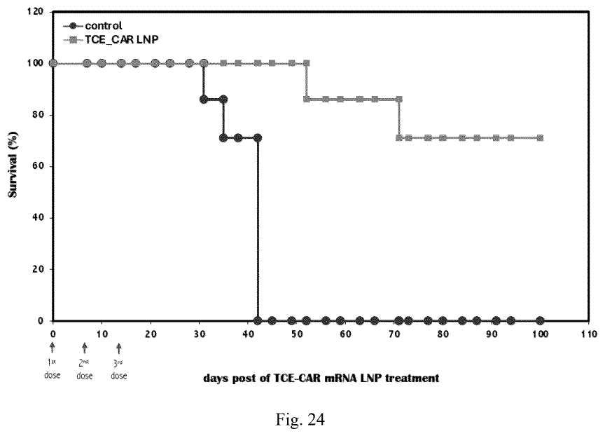

Tumor size measurements from individual animals are shown in FIG. 22. Remarkably, 71% (5/7) of animals treated with TCE^{SR4}_CAR^{SR9} mRNA LNP showed no tumor growth or a reduction in tumor size, i.e., cancer-free, up to 100 days after the first dose. See also FIG. 24.

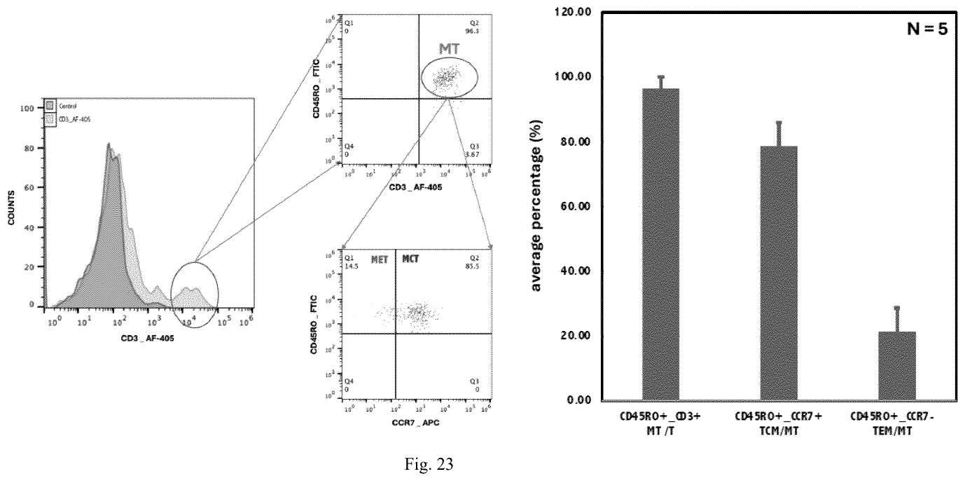

The spleen cells collected from the cancer-free mice (FIG. 22) were analyzed using flow cytometry assay. The results are shown in FIG. 23. The majority (>90%) of human T cells isolated from the murine spleens were MT cells, with ~80% being TCM. This result strongly suggests that these memory T cells contribute to the long-term cancer-free survival of the TCE^{SR4}_CAR^{SR9} mRNA LNP treated animals.

### Example 7: The Lead TCE_CAR mRNA LNP has No Detectable Toxicity

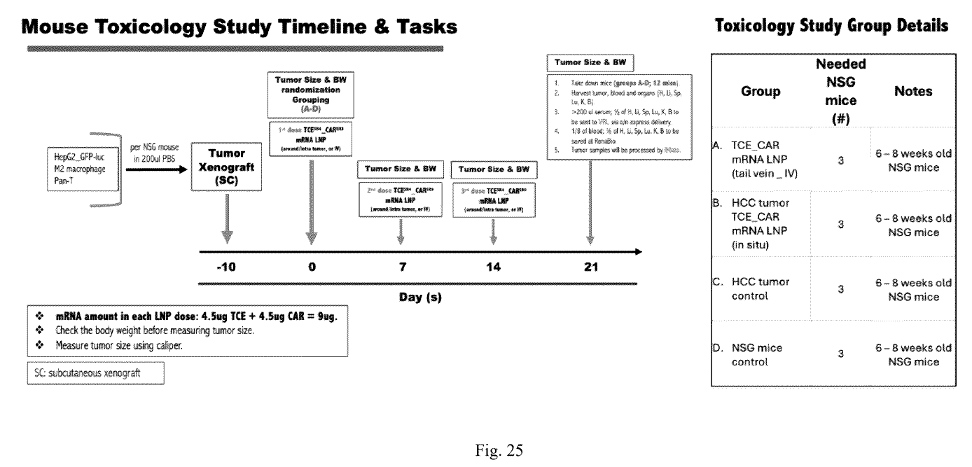

In vivo toxicology studies were performed in the murine model for HCC described in Example 6 according to the design shown in FIG. 25. The study was essentially identical to the in vivo efficacy study except that animals were randomized to 4 groups as shown in FIG. 25, and the study was terminated after 21 days post-treatment.

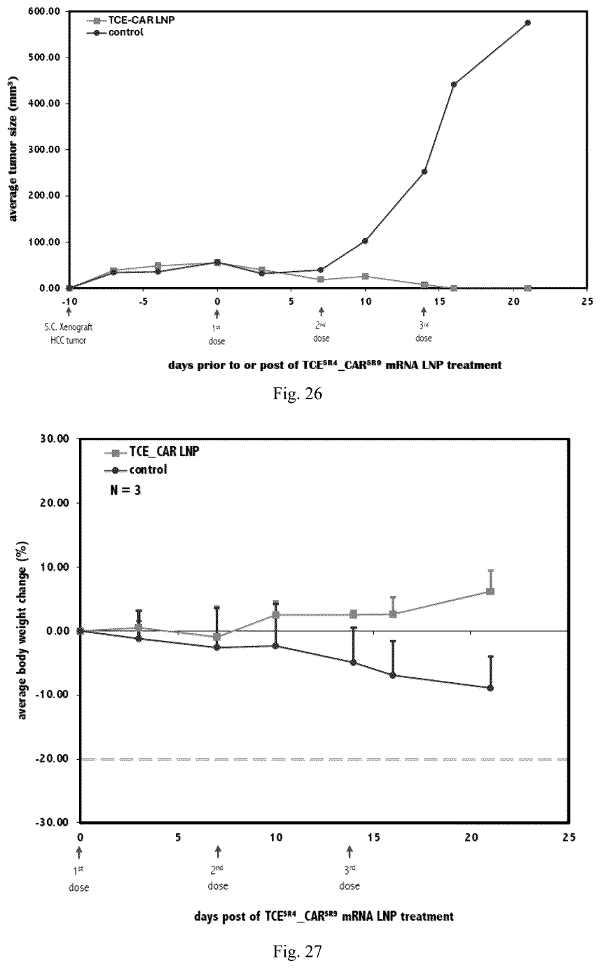

Measurements of average tumor size (FIG. 26) and body weight (FIG. 27) confirmed the results from the efficacy study described in Example 6.

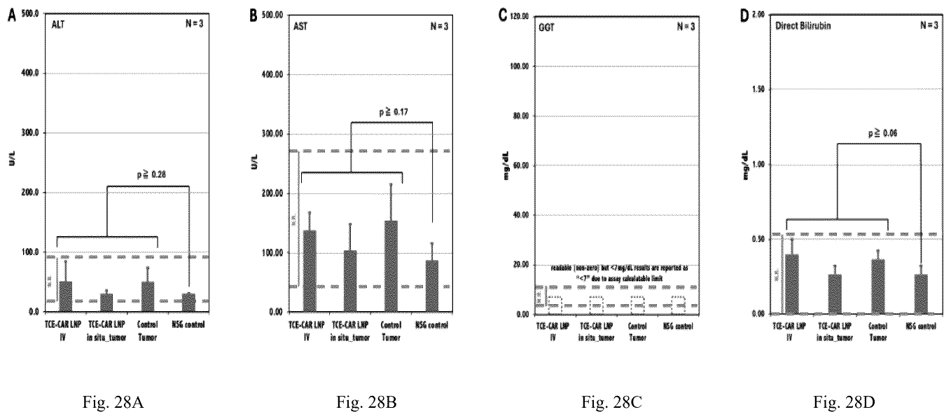

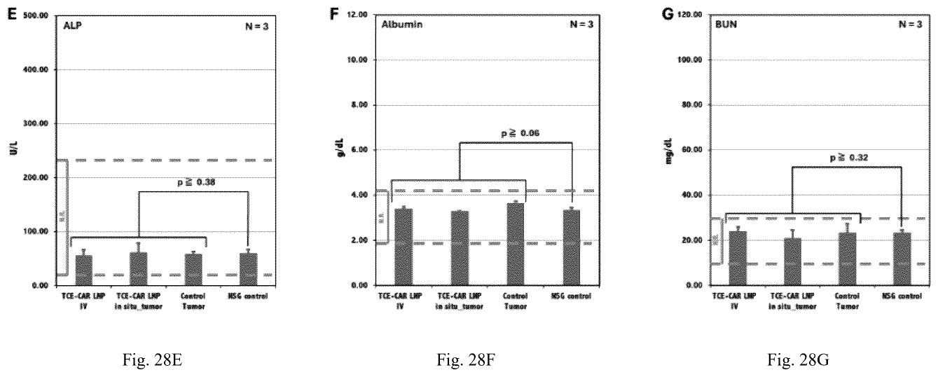

Toxicity of the TCE^{SR4}_CAR^{SR9} mRNA LNP treatment was assessed by examining the serum levels of liver and kidney biomarkers. The results are shown in FIGS. 28A-28G. No liver or kidney toxicity was detected in animals injected intravenously or intratumorally with the TCE^{SR4}_CAR^{SR9} mRNA LNP.

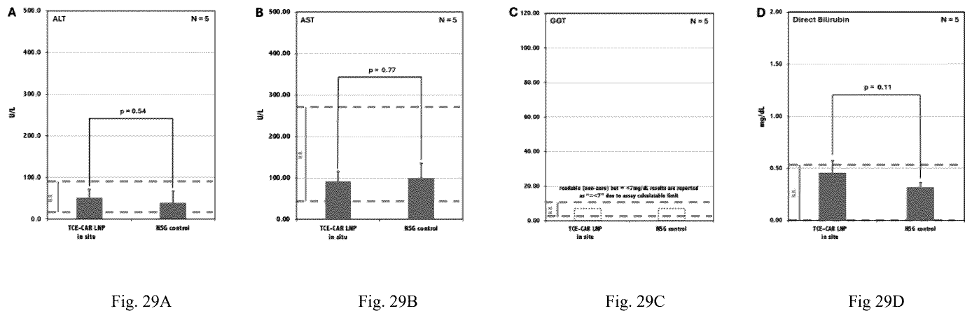

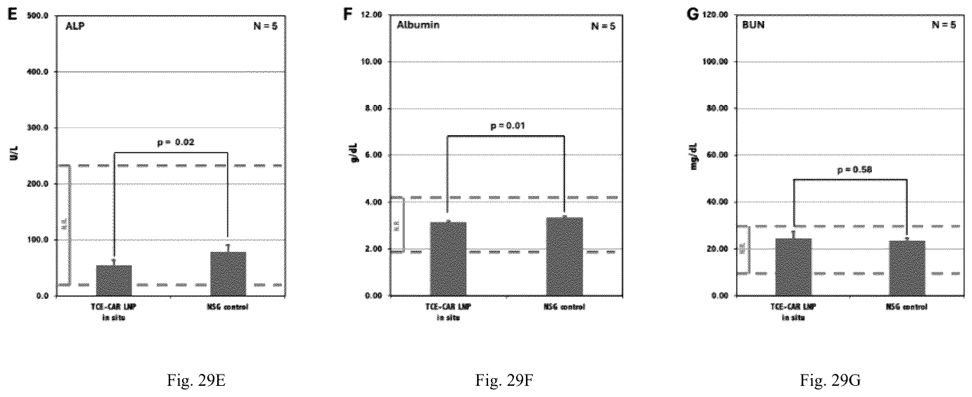

Serum samples from the cancer-free animals described in Example 6 were also analyzed for liver and kidney biomarkers. The results, shown in FIGS. 29A-29G, also show no toxicity of the TCE^{SR4}_CAR^{SR9} mRNA LNP treatment.

Patho-histological examination of major organs from the experimental animals confirmed that there were no major adverse effects of the TCE^{SR4}_CAR^{SR9} mRNA LNP treatment. The results are summarized below in Tables 4 and 5.

TABLE 4
Histologic scoring of organs from experimental animals

| | | | | RNB-001 | | | | | | | | |
|---|---|---|---|---|---|---|---|---|---|---|---|---|
| | | | | HCC tumor | | | HCC tumor | | | | | |
| | RNB-001 (IV) | | | (in situ) | | | control | | | Normal NSG | | |
| Group | #1 | #2 | #3 | #1 | #2 | #3 | #1 | #2 | #3 | #1 | #2 | #3 |
| Heart | N | N | N | N | N | N | N | N | N | N | N | N |
| Liver | N | N | N | N/2MF | N/2MF | N | 3D | N | 3D | N | N | N |
| Spleen | N | N | N | N | N | N | N | N | N | N | N | N |
| Lung | N | N | N | 3D | 4D | N/2MF | 4D | N | 4D | N | N | N |
| Kidney | N | N | N | N | N | N | 3D | N | N/2MF | N | N | N |
| Brain | N | N | N | N | N | N | N | N | N | N | N | N |

2 = Mild
3 = Moderate
4 = Marked
N = Normal
MF = Multifocal
D = Diffuse

TABLE 5
Histologic scoring of organs from cancer-free animals

| | RNB-001 HCC tumor (in situ) | | | | | Normal NSG (control) | | | | |
|---|---|---|---|---|---|---|---|---|---|---|
| Group | #1 | #2 | #3 | #4 | #5 | #1 | #2 | #3 | #4 | #5 |
| Heart | N | N | N | N | N | N | N | N | N | N |
| Liver | N | N | N | N | N | N | N | N | N | N |
| Spleen | N | N | N/3F | N | N | N | N | N | N | N |
| Lung | N/2MF | N/2F | N | N | N | N | N | N | N | N |
| Kidney | N/1MF | N | N | N | N | N | N | N | N | N |
| Brain | N | N | N | N | N | N | N | N | N | N |

1 = Minimal
2 = Mild
3 = Moderate
N = Normal
MF = Multifocal
F = Focal

The observation of nodules of human lymphocytes and macrophages was as expected, which resulted from injection of human immune cells circulating in the NSG mice. Pathologies observed were: Liver, small nodules of lymphocytes and macrophages in perivascular connective tissue, primarily adjacent to central veins; Lung, small nodules of lymphocytes, cell debris and macrophages surrounding blood vessels, generally arterioles; Kidney, small nodules of lymphocytes, cell debris and macrophages throughout the cortex, corticomedullary area, medulla, and suburothelial pelvis.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A nanoparticle for treating hepatocellular carcinoma (HCC), the nanoparticle comprising a lipid phase and a core, the core comprising one or more nucleic acids that encode a chimeric antigen receptor (CAR), a T-cell engager (TCE), and, optionally, a therapeutic gene, wherein the CAR and the TCE each bind specifically to an HCC tumor-associated antigen and the nanoparticle selectively delivers the one or more nucleic acids to M2 macrophages in vitro and in vivo.

2. The nanoparticle of claim 1, wherein the lipid phase includes:
an ionizable lipid;
cholesterol or its derivative;
a phospholipid; and
a polyethylene glycol-conjugated lipid (pegylated lipid), and the core further comprises a buffer solution.

3. The nanoparticle of claim 2, wherein a mole percentage of the ionizable lipid, the cholesterol or derivative, the phospholipid, and the pegylated lipid in the nanoparticle is 37% to 47%, 42% to 50%, 7% to 12%, and 1.5% to 5.5%, respectively, and a molar ratio of the ionizable lipid to the one or more nucleic acids is 2:1 to 6:1.

4. The nanoparticle of claim 2, wherein the ionizable lipid is selected from D-Lin-MC3-DMA, SM102, LP01, ALC-0315, and analogs thereof.

5. The nanoparticle of claim 2, wherein the phospholipid is selected from DLPC, DPPC, DSPA, DPPA, DSPC, DMPE, DHPC, DMPC, SPPC, and DAPC.

6. The nanoparticle of claim 5, wherein the pegylated lipid is selected from DSPE-PEG 2000, DMG-PEG-2000, DSPE-PEG 2000-Mannose, and DMG-PEG-2000-Mannose.

7. The nanoparticle of claim 6, wherein the lipid phase includes at least two pegylated lipids.

8. The nanoparticle of claim 1, wherein the CAR binds specifically to GPC-3, the TCE binds specifically to an HCC TAA and to CD3, and the therapeutic gene, if present, is an anti-VEGF antibody.

9. The nanoparticle of claim 8, wherein the CAR has the amino acid sequence of any one of SEQ ID NOs 6 to 9.

10. The nanoparticle of claim 9, wherein the TCE has the amino acid sequence of any one of SEQ ID NOs 1 to 5.

11. The nanoparticle of claim 10, wherein the CAR has the amino acid sequence of SEQ ID NO: 9 and the TCE has the amino acid sequence of SEQ ID NO: 4.

12. A therapeutic composition for treating HCC, comprising a plurality of the nanoparticle of claim 1 and a pharmaceutically acceptable excipient.

13. A method for converting HCC tumor-resident M2 macrophages into M1 macrophages, the method comprising contacting the M2 macrophages with a plurality of the nanoparticle of claim 1.

14. A method for treating HCC in a subject, the method comprising administering to the subject a composition comprising the nanoparticle of claim 1, wherein the CAR specifically binds to GPC-3, the TCE specifically binds to EGFR, EpCAM, or both, and binds to a T cell, and the therapeutic gene, if present, is effective against HCC.

15. The method of claim 14, wherein the CAR has the amino acid sequence of any one of SEQ ID NOs 6 to 9.

16. The method of claim 15, wherein the TCE has the amino acid sequence of any one of SEQ ID NOs 1 to 5.

17. The method of claim 14, wherein the CAR has the amino acid sequence of SEQ ID NO: 9 and the TCE has the amino acid sequence of SEQ ID NO: 4.

18. The method of claim 14, wherein the lipid phase comprises a pegylated lipid selected from DSPE-PEG 2000, DMG-PEG-2000, DSPE-PEG 2000-Mannose, DMG-PEG-2000-Mannose, and mixtures thereof.

19. The method of claim 18, wherein the pegylated lipid is DMG-PEG-2000.

20. The method of claim 14, wherein the therapeutic gene increases the anti-cancer efficacy of the composition, as compared to the combined anti-cancer efficacy of the CAR and TCE.

21. A chimeric antigen receptor (CAR) for treating HCC, the CAR having the amino acid sequence selected from SEQ ID NOs 6 to 9.

22. An isolated nucleic acid encoding the CAR of claim 21.

23. A T Cell Engager (TCE) for treating HCC, the TCE having the amino acid sequence selected from SEQ ID NOs 1 to 5.

24. An isolated nucleic acid encoding the TCE of claim 23.


SEQUENCE LISTING (10 sequences)

SEQ ID NO: 1 - protein, 652 aa, synthetic construct
  source 1..652: note = SR1
METDTLLLWVLLLWVPGSTGDQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSHHHHHH

SEQ ID NO: 2 - protein, 652 aa, synthetic construct
  source 1..652: note = SR2
METDTLLLWVLLLWVPGSTGDELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 3 - protein, 657 aa, synthetic construct
  source 1..657: note = SR3
METDTLLLWVLLLWVPGSTGDEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSHHHHHH

SEQ ID NO: 4 - protein, 657 aa, synthetic construct
  source 1..657: note = SR4
METDTLLLWVLLLWVPGSTGDELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKGGGGSEVQLVESGGGLVQAGGSLRLSCAASGRTFSSYVMGWFRQATGKEREFVATIAWDSGSTYYADSVKGRFTISRDNAKNTVHLQMNSLKPEDTAVYYCAASYNVYYNNYYYPISRDEYDYWGQGTQVTVSSHHHHHH

SEQ ID NO: 5 - protein, 523 aa, synthetic construct
  source 1..523: note = SR5
METDTLLLWVLLLWVPGSTGDELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 6 - protein, 447 aa, synthetic construct
  source 1..447: note = SR6
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 489 aa, synthetic construct
  source 1..489: note = SR7
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 591 aa, synthetic construct
  source 1..591: note = SR8
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 9 - protein, 633 aa, synthetic construct
  source 1..633: note = SR9
MALPVTALLLPLALLLHAARPDVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRNIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKAL

SEQ ID NO: 10 - DNA, 2519 bp, synthetic construct
  source 1..2519: mol_type = other DNA; note = mRNA IVT plasmid backbone
ggtttagtgaaccgtcagatccgctagagatccgcggccgctaatacgactcactataaggacatttgcttctgacacaactgtgttcactagcaacctcaaacagacaccgctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaccggtctagcataaccccttggggcctctaaacgggtcttgaggggttttttgcccgggggatcccctagggcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttaaacggaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaaatgcggcctcgagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtt